(19) United States
(12) Patent Application Publication
(10) Pub. No.: US 2013/0305454 A1
(43) Pub. Date: Nov. 21, 2013

(54) APPARATUS FOR TRANSPORTING A WOUNDED PERSON

(71) Applicant: Peleg Ben-Galim, Houston, TX (US)

(72) Inventor: Peleg Ben-Galim, Houston, TX (US)

(73) Assignee: MOR RESEARCH APPLICATIONS LTD., Tel-Aviv (IL)

(21) Appl. No.: 13/982,287

(22) PCT Filed: Jan. 26, 2012
(86) PCT No.: PCT/IB2012/050377

§ 371 Date: Jul. 29, 2013

Related U.S. Application Data
(60) Provisional application No. 61/437,423, filed on Jan. 28, 2011.

(51) Int. Cl. A61B 17/132; A61G 1/00

(52) U.S. Cl. CPC A61B 17/1325; A61G 1/00
USPC 5/628; 606/203

(57) ABSTRACT

Sleeves for immobilizing and compressing one or more limbs of a patient are described. Sleeve embodiments can be rapidly applied to a wounded limb, and will limit bleeding from the limb as well as rapidly immobilizing the limb in a manner appropriate for transporting the patient. A stretcher equipped with multiple sleeves for full-body immobilization and to control bleeding in situations of multiple traumas is described. A method for controlling bleeding from a pelvic wound is also presented.

### RELATED APPLICATION

This application claims priority from U.S. Provisional Application No. 61/437,423, filed Jan. 28, 2011. The contents of that application are incorporated by reference as if fully set forth herein.

### FIELD AND BACKGROUND OF THE INVENTION

The present invention, in some embodiments thereof, relates to an immobilization device for protecting and transporting an injured patient, and more particularly, but not exclusively, to a device for transporting and protecting a patient with injured limbs, pelvic wounds, and other forms of bleeding trauma.

Devices for stabilization and transportation of injured patients are known in the art. Automobile accidents, industrial accidents, and acts of war and of terrorism unfortunately provide many situations in which individuals are badly hurt, their bodies badly damaged, often with multiple fractures and multiple open wounds. The present invention, in some embodiments thereof, particularly but not exclusively addresses situations in which fractures or other wounds to one or several of the patient's limbs must be stabilized simultaneously and/or in which bleeding from several sites must simultaneously be controlled.

Often, for example in major road accidents and in military emergencies, medical personnel have a need for rapid and effective control of bleeding from multiple body sites, a need to reduce fractures or at least stabilize broken bones in several limbs of a patient at the same time, and a need to immobilize the body as a whole, all of these needing to be handled as rapidly as possible so as to enable immediate transport of the patient to a hospital or other treatment site, while yet minimizing dangers to the patient resulting from the immobilization processes, the control of bleeding processes, and the transportation processes themselves.

Extrication stretchers, pressure dressings and limb fixation devices are generally known in the art. These devices are typically designed for one-time use and are carried on ambulances and other emergency vehicles.

Combat patients (e.g. soldiers) and civilian victims of motor vehicle accidents, falls, natural catastrophes and terrorist activities often suffer from multiple injuries ranging from obvious external injuries to non-visible internal injuries that can be the greatest cause of morbidity and mortality. Many of these internal injuries will not be definitively diagnosed until the patient/soldier reaches the hospital. During the processes by which the patient is extracted from the scene of the trauma and transported to the hospital, some of these injuries can be exacerbated, even to the point of killing the patient. Therefore speed of evacuation (e.g. from a battlefield scene) can be crucial to survival of a patient, but rapid stabilization of the injured patient and effective control of bleeding must be accomplished prior to moving the patient to a medical facility. Patients suffering from multiple trauma injuries need to be handled both rapidly and effectively to prevent loss of life.

Background art which might be considered relevant to this field includes the following patent documents: U.S. Pat. No. 3,933,150, GB1384278, U.S. Pat. No. 2,449,767, U.S. Pat. No. 2,377,940, U.S. Pat. No. 2,511,061, U.S. Pat. No. 3,139,883, FR2583977(A1), U.S. Pat. No. 7,389,553, EP1214919(A1), JP9154888, AND EP0301614(A1).

### SUMMARY OF THE INVENTION

According to an aspect of some embodiments of the present invention there is provided a device for stabilizing, stretching, and compressing a wounded limb of a patient, comprising

a) an elastic net which comprises
i) a plurality of lateral fasteners which, when fastened, shape the net into a sleeve which may be fastened around the limb;
ii) at least one proximal fastener on a proximal end of the sleeve; and
iii) at least one distal fastener on a distal end of the sleeve,

the net being so constructed that when a limb is encased in the net by fastening the lateral fasteners and longitudinal traction of between 5 kg and 20 kg is applied to the net by pulling the proximal and the distal fasteners away from each other, sufficient lateral pressure is applied toward the limb by the net to reduce bleeding from the limb.

According to some embodiments of the invention, the device further comprises a framework sufficiently strong to support at least a limb of a body, and to which the proximal and digital fasteners may be attached.

According to some embodiments of the invention, the device further comprises a tension device for providing a traction tension by pulling the proximal and the distal fasteners away from each other, the tension being adjustable to a range at least partly overlapping with the range of between 5 kg and 20 kg of tension.

According to some embodiments of the invention, the device is a stretcher.

According to some embodiments of the invention, the device further comprises four of the nets, two sized to form sleeves each sized accommodate an arm, and two sized form sleeves each sized to accommodate a leg.

According to some embodiments of the invention, the stretcher comprises at least one extended handle whose length is adjustable.

According to some embodiments of the invention, the framework comprises a Thomas splint.

According to an aspect of some embodiments of the present invention there is provided a full-body transportation apparatus for transporting a wounded patient, which comprises

a) a stretcher which comprises a pelvic attachment for attaching a pelvis of a patient to the stretcher;

b) a plurality of nets each of which comprises
i) an elastic body;
ii) lateral fasteners which shape the material into a sleeve which may be fastened around a limb of a patient;
iii) proximal and distal fasteners attachable to portions or fixtures of the stretcher; and
iv) a tension controller operable to introduce a controllable degree of longitudinal traction to the each sleeve,

and wherein at least some sleeves formed of the nets are so constructed that introducing longitudinal traction forces on at least one sleeve formed from at least one of the nets produces lateral constricting pressures on a limb contained within the sleeve.

According to some embodiments of the invention, the stretcher further comprises a pressure sensor for measuring longitudinal traction applied to at least one of the nets formed as a sleeve and enclosing a limb of a patient, and may optionally comprise a pressure sensor for measuring lateral constricting pressure applied to an enclosed limb by at least one of the nets formed as a sleeve, and may optionally comprise a pulse sensor operable to detect and report strength of a pulse on a distal portion of a limb under traction by one of the nets formed as a sleeve and enclosing the limb, and may optionally comprise a blood oxygenation detector operable to detect blood flow status on a distal portion of a limb under traction by one of the nets formed as a sleeve and enclosing the limb.

According to some embodiments of the invention, the stretcher comprises telescoping handles each of which may be fixed to be of a selected length, the handles comprising a feature to which a distal attachment of one of the nets may be attached.

According to some embodiments of the invention, the lateral constricting pressures on a limb contained within a sleeve is sufficient to reduce bleeding from the limb.

According to an aspect of some embodiments of the present invention there is provided a method for reducing a fracture of a limb, comprising

a) enclosing the fractured limb in an net; and

b) creating a traction tension by pulling a distal end of the net away from a proximal end of the net, thereby elongating the net, causing the net to apply pressure to the enclosed limb and to transfer at least some of the traction to the limb, thereby reducing the fracture.

According to some embodiments of the invention, the pressure applied by the net to the enclosed limb is sufficient to reduce bleeding from the limb.

According to some embodiments of the invention, the traction tension is regulated to a tension of between 5 kg and 20 kg.

According to some embodiments of the invention, the method further comprises using a pressure sensor to report traction tension induced in the net.

According to some embodiments of the invention, the method further comprises adjusting traction tension in the net so as to maximize tension without reducing circulation in the limb to a dangerous degree.

According to some embodiments of the invention, the method further comprises utilizing one of a pulse detector and a blood oxygenation detector to detect dangerous reduction in blood circulation of the limb.

According to an aspect of some embodiments of the present invention there is provided a method for controlling bleeding from a pelvic wound of a patient during transportation of the patient, comprising:

a) applying bandaging materials in or on the pelvic wound;

b) attaching the patient to a framework having a proximal end of an elastic material attached to the framework near a midline of the framework and near a feature designed to receive and bind a pelvis of a patient to the framework;

c) positioning a medial portion of the material so that it at least partially covers the bandaging material;

d) attaching a distal end of the elastic material to the framework in a manner which causes the material to substantially cover the bandages, cross over the patient's body, pass near an upper pelvic brim of the patient, and be attached to the framework; and

e) inducing a longitudinal tension in the material, thereby pressing the bandaging material toward the pelvic wound to inhibit bleeding therefrom.

According to an aspect of some embodiments of the present invention there is provided a method for reducing bleeding from a wounded limb, comprising

a) enclosing the wounded limb in an net; and

b) creating a traction tension by pulling a distal end of the net away from a proximal end of the net, thereby elongating the net, causing the net to apply pressure to the enclosed limb, the pressure being sufficient to reduce bleeding from the limb.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a simplified schematic showing open and closed configurations of a protective sleeve for immobilizing a limb of a wounded patient, according to an embodiment of the present invention;

FIG. 1B is a simplified schematic showing an exemplary embodiment of the sleeve of FIG. 1A, according to an embodiment of the present invention;

FIG. 2 is a simplified schematic showing a stretcher with multiple sleeves for immobilizing and protecting all four limbs of a patient, according to an embodiment of the present invention. In FIG. 2 the stretcher is shown with open and empty sleeves, ready for use;

FIG. 3 is a is a simplified schematic showing the stretcher of FIG. 2, closed upon and holding a patient ready for transport, according to an embodiment of the present invention;

FIG. 4 is a simplified schematic showing a method for using a sleeve to control bleeding from a limb stump or other open wound or cavernous bleeding site of a pelvis; and

FIG. 5 is a flowchart of the method of claim 4, according to an embodiment of the present invention.

### DESCRIPTION OF SPECIFIC EMBODIMENTS OF THE INVENTION

The present invention, in some embodiments thereof, relates to an immobilization device for protecting and transporting an injured patient, and more particularly, but not exclusively, to a device for transporting and/or protecting a patient with injured limbs, and/or for reducing limb fractures, and/or for controlling bleeding in a wounded patient.

In some embodiments an immobilization and protection device comprises a sleeve which can straighten, stretch, immobilize and protect an injured limb, constraining the limb in a position where it is unlikely to sustain further damage during transportation of the patient, and which sleeve also and simultaneously provides a controlled surrounding pressure to that limb, thereby helping to control bleeding from damaged limb tissue.

In some embodiments the sleeve is formed of a woven net of fibers which may be placed or fastened so as to surround a wounded limb. Optionally, the sleeve net is constructed of a durable but elastic material. In some embodiments the sleeve material is sufficiently elastic and can be so fastened around a limb that the elastic properties of the material create lateral pressure on the limb, limiting bleeding, and optionally holding pressure bandages in place. In some embodiments the net of fibers is so constructed that when closed over a limb it exerts a centripetal force (i.e. a surrounding lateral pressure) on the contained limb, and that force is augmented when a longitudinal tension is placed on the sleeve. In some embodiments, surrounding lateral pressure on the enclosed limb is created both by lateral elasticity of the sleeve and also by the lateral compression created by longitudinal traction. In such embodiments, by fastening the net around the limb and also stretching the net longitudinally, the limb is thereby straightened, immobilized, and also subjected to a constricting centripetal pressure which serves to at least partially control bleeding from the limb and to reduce the blood content of the enclosed limb. The amount of centripetal pressure applied is a function of the tension used in stretching the net, and is thus controllable.

Longitudinal tension on the sleeve also tends to reduce fractures and fracture dislocations and holds them in an aligned position and stabilizes them to the limb and to the body and (optionally) to the framework.

By using such a sleeve, a simple enveloping and stretching operation which can be performed simply and rapidly may accomplish some or all of these goals: a) it positions the limb in a physiologically desirable configuration, b) it stabilizes the limb for transport, c) it reduces and stabilizes fractures, and d) it subjects the limb to a controlled constricting pressure which serves to reduce bleeding.

In some embodiments a single sleeve is provided and can be used for treating a single injured limb. In some embodiments, including embodiments appropriate for secure transport of multiply wounded patients to a medical facility, multiple sleeves are provided for containing up to all four limbs of a patient, each sleeve having a proximal end attached to a stretcher, the sleeves being used in combination with means for attaching a patient's pelvis, shoulders, and head to the stretcher, so that the entire body including up to all four limbs of the patient is stabilized and prepared for transport.

In some additional embodiments presented below, an elastic material is stretched over an amputation stump or a cavernous pelvic wound, to control bleeding therefrom.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

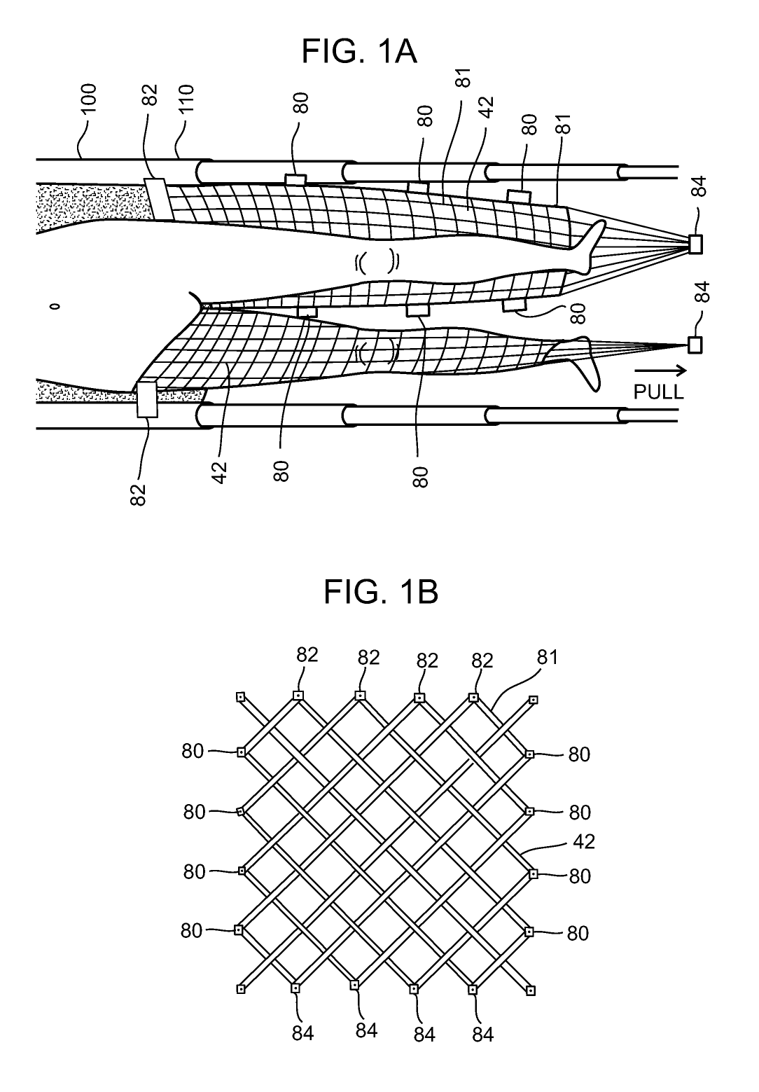

Referring now to the drawings, FIG. 1A is a simplified schematic showing open and closed configurations of a protective sleeve for immobilizing a limb of a wounded patient, according to an embodiment of the present invention.

FIG. 1A shows the lower half of the body of a patient lying on a framework 100 embodied (in this exemplary embodiment) as a stretcher 110. The left leg of a patient (upper leg in the figure) is shown lying on a net 81, which is optionally constructed of an elastic or semi-elastic material. Net 81 is optionally provided with lateral attachments 80 (also referred to herein as “fasteners 80” by which net 81 may be closed upon itself to form a sleeve configuration herein labeled sleeve 42. Lateral attachments 80 may be formed as zippers, as Velcro connections, as tie strings, or may be any other method of tying or attaching the material of net 81 to form sleeve 42 around a limb in such a way that the limb is largely or completely contained within the sleeve. Attachments 80 may optionally be extended in length (for example, a zipper) or may optionally be provided in units separated one from another by a distance of between 5 mm and 8 cm. When used in context of a stretcher used for emergency transportation of badly wounded patients, connections 80 are optionally designed to be attachable rapidly and simply, and the number of attachments used is optionally selected to provide a connection which is sufficient to hold the limb yet sparse enough to enable rapid opening and closing of the sleeve. For example, optionally a minimum of one pair of connectors 80 (e.g. a zipper) may be used, or 2, 3, 10, or up to 30 pairs of connectors may be used. Optionally, attachments 80 may themselves be elastic and allow elastic attachment so as to introduce a degree of pressure of sleeve 42 on the contained limb when attachments 80 are attached. For example, attachments 80 configured as a plurality of Velcro strips attached to an elastic material can provide some or all of rapid closing, rapid release, lateral pressure applied to the limb by pulling the net while closing attachments 80, and possibilities of partially releasing a selected portion of sleeve 42 (by opening selected attachments 80, e.g. to change a bandage) while leaving other portions of sleeve 42 connected.

Optionally, net 81 is a net of woven or knotted otherwise connected fibers. Spacing between elements of net 81 is optionally between one millimeter and three centimeters, for example 5 mm or 1 cm.

In some embodiments sleeve 42 is designed to extend from near a patient's shoulder or armpit, and to extend beyond the hand to an attachment point on a framework. Accordingly, in some embodiments sleeve 42 designed for an arm of an adult will be between 60 cm and 120 cm in length prior to the application of traction, and traction will cause it to stretch by between 5% and 100% depending on materials used. In some embodiments a sleeve for an adult leg will be between 70 and 160 cm in length. In some embodiments, circumference of the proximal opening of a sleeve 42 will be between 1.5 times bigger than the circumference of the distal opening of the sleeve and 15 times bigger than the distal opening of the sleeve.

In an alternative design useful for in-hospital treatment of patients, sleeve 42 may optionally be fabricated as a closed sleeve, and slipped over a patient's limb. Such a closed sleeve may optionally comprise straps or elastic elements enabling to tighten it.

The right leg of the patient in FIG. 1A (the lower leg on the figure) is shown already substantially enclosed in a sleeve 42.

Sleeve 42 is optionally provided with one or more proximal fasteners 82 (at one longitudinal end of sleeve 42) and/or one or more distal fasteners 84 (at the other longitudinal end of sleeve 42). Proximal fasteners 82 and distal fasteners 84 may be attached to a stretcher, framework, or to a stable part of a patient's body, and traction may applied to a limb contained within sleeve 42 by pulling proximal and distal fasteners 82 and 84 in opposite directions, or by immobilizing one set of fasteners (proximal or distal) and by pulling the other set of fasteners away from the immobilized set. Sleeve 42 is characterized in that when sleeve 42 is wrapped around a limb and then stretched longitudinally, there results a centripetal pressure of sleeve 42 on a limb contained therein. Typically, a traction tension of between 5 kg and 20 kg (for example, around 10 kg) will be used, though clinical considerations in any particular case may dictate use of more or less tension. In some method of use embodiments, when control of bleeding is required, traction pull is increased until just before loss of pulse in the lower limb (or impaired oxygenation of the limb) is detected. Optionally, in case of life-threatening bleeding, tension may be temporarily increased to beyond the point where loss of pulse or insufficient oxygenation are detected.

An optional method of construction of sleeve 42 is shown in FIG. 1A, where individual cords of net 81 run directly from proximal connectors 82 to distal connectors 84, so that pulling proximal and distal connectors away from each other creates a centripetal force on net 81, and a surrounding lateral force on a limb enclosed by the net. An alternative configuration for net 81 and sleeve 42 is shown in FIG. 1B, which is a simplified schematic showing a portion of an exemplary embodiment of sleeve 42, according to an embodiment of the present invention. The weave shape shown in FIG. 1B is somewhat similar to the “Chinese finger trap” toys that sometimes appear at children's parties. Both the configuration shown in FIG. 1A and the configuration shown in FIG. 1B are such that if longitudinal traction is applied to a sleeve 42 surrounding a limb, (e.g. by a traction mechanism which pulls distal attachments 84 and proximal attachments 82 away from each other), lateral (centripetal) pressure will be exerted by sleeve 42 on the enclosed limb. Both the weave structure shown in FIG. 1B and the alternate configuration shown in FIG. 1A should be understood to be exemplary only, and not limiting. Any weave or other construction which results in lateral (centripetal) pressure on the limb when the distal and proximal ends of sleeve 42 are pulled away from each other can be used.

Optionally, straps or elastic materials may be provided to create or enhance lateral pressure on the limb.

As sleeve 42 is stretched longitudinally, the material of which sleeve 42 is constructed applies pressure laterally to the contained limb. Because of the weaved construction and elasticity of the materials, such pressure tends to distribute itself throughout the surface, providing a relatively even constricting pressure over the limb.

This lateral pressure combines with pressures exerted by the longitudinal traction to limit blood loss from the wounded limb. Restriction of blood flow due to elongation of the limb under traction, and restriction of blood flow due to lateral compression of the limb under conditions of longitudinal elongation, optionally combine to protect the patient from excessive blood loss from open exanguinating wounds and open fractures during immobilization of the limb and transportation of the patient. Another potential advantage of the use of sleeve 42 is that it assists the body's physiological reaction to shock by reducing the amount of blood held in the limbs, thereby concentrating blood in the critical regions (brain, heart, lungs, etc.) through lateral surrounding pressure exerted on the limbs by sleeves 42, which increases the peripheral vascular pressure in the limbs.

In some embodiments, use of longitudinal traction and lateral constriction will reduce blood loss by at least 50%. In cases of catastrophic damage (a near amputation for example, with severing of important arteries, pressure may be increased to reduce bleeding by amounts in excess of 95%.

Compresses, pressure bandages or other bandages, or other suitable materials may optionally be positioned on the limb and within sleeve 42, on open wounds or at other strategic positions where heightened pressure is desired. Balls or other space-taking objects may also optionally be so used to enhance pressure, e.g. above an artery. Such compresses or other objects can optionally be positioned on the wounded limb prior to closing net 81 to form sleeve 42. With such compresses or pads or bandages in place, applying longitudinal stretching to sleeve 42 presses these pads or bandages onto the limb where they can further enhance control of bleeding from the contained limb. Once longitudinal traction is applied to sleeve 42, sleeve 42 is pressed towards the bandages and serves to hold them in place over the wounds or at other selected positions. Individual fasteners 80 can be opened as needed to allow changes of bandages or other treatments to the limb.

Optionally, sleeve 42 (and net 81) may be formed to roughly correspond to the shape of the limb (arm, leg) it is designed to hold. When longitudinal tension is applied, sleeve 42 closes itself around the detailed form of the specific limb contained. Optionally, a selection of basic sleeve sizes may be provided (e.g. in a kit), so as to accommodate major differences in body form (e.g. so that a sleeve appropriate for the leg of an elderly lady need not be used on a large male football player). Optionally, lateral fasteners 80 can be elastic and/or adjustable, so as to enable a paramedic to establish lateral pressure surrounding the limb independent of (or prior to) application of longitudinal traction.

Sleeve 42 can optionally be used together with any frame which enables attachment of the sleeve to the limb and application of traction as required to immobilize the limb and provide lateral pressure through longitudinal traction as described above. In an exemplary embodiment of the invention the frame used should be strong enough to support the limb (or the entire body, if a whole-body frame such as a stretcher is used). Optionally, attachment points on the frame are provided for attaching the body and/or for attaching proximal and distal attachments (82 and 84) of sleeve(s) 42. Optionally, the frame is transparent to x-rays (being constructed of carbon-fiber materials, for example) so as to enable x-ray and CT examination of the patient without removing the patient from the frame. Optionally, radio-opaque markers, for example left-right markers and size markers, may be used with or incorporated into a radio-transparent frame.

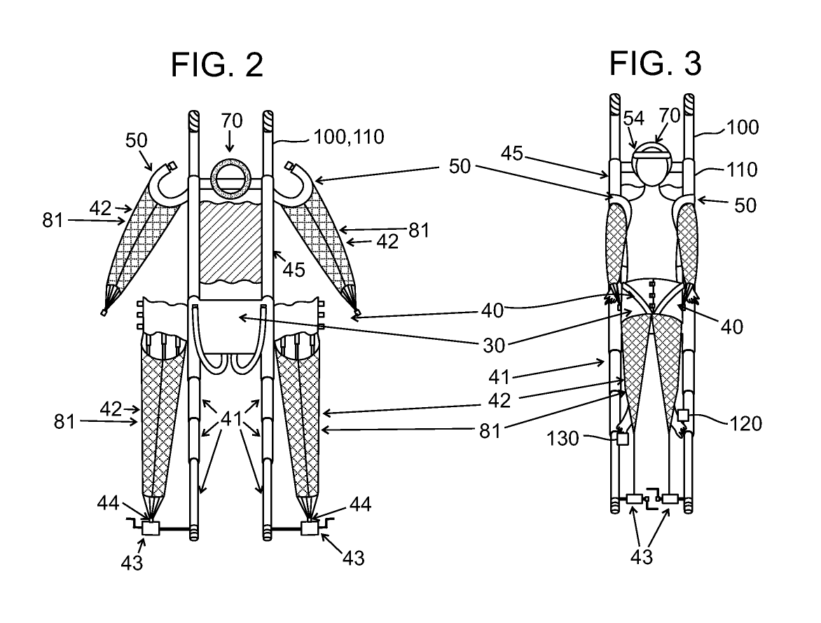

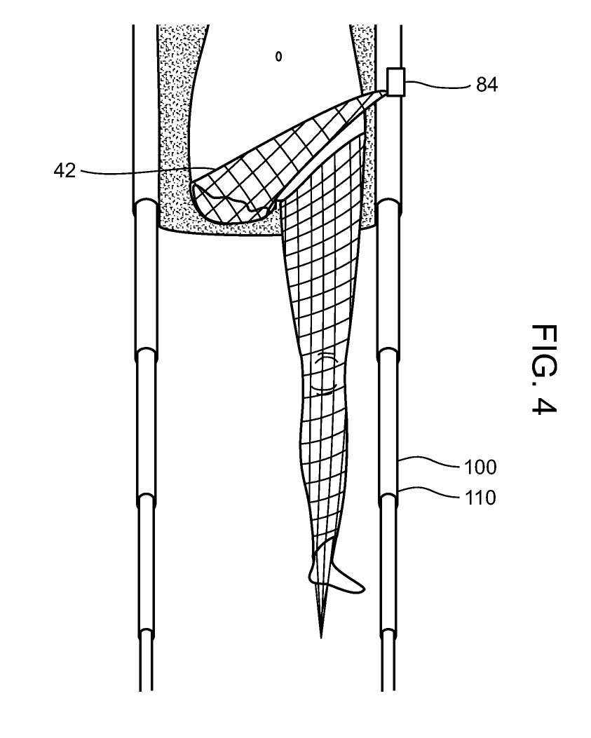

Optionally, the frame can provide a ratchet and/or pulley system (e.g. element 43 on FIGS. 2 and 3) can provide traction to sleeve(s) 42. For example, a proximal end of such a frame might be provided with a pelvic strap (30 and 40 in FIGS. 2 and 3) to attach the frame to a pelvis, and a distal portion of the frame could extend below a patient's foot so as to provide a framework for stretching a sleeve 42 between distal and proximal portions of the framework, as shown in FIGS. 2 and 3.

In another example, a Thomas splint frame (comprising a ring sized to accommodate the diameter of a proximal portion of a limb joined to an elongated rectangular frame) may be used to hold a single limb in traction: A sleeve 42 is stretched between the ring of the Thomas splint frame (pushed around the leg of the patient and abutting his pelvis) and the distal end of the Thomas splint frame, the frame itself being placed alongside the leg.

Optionally, sleeve 42 can be shaped so that the distal side of the net is more narrow than the proximal side. Optionally, angles of fibers of net 81 is different at different portions of the sleeve 42, so as to create a desired profile of stretch and pressure ratios at different parts of the sleeve.

Optionally, sleeve 42 comprises an elastic material and/or a knit and/or a mesh and/or a solid material which comprises spiral wires.

Attention is now drawn to FIGS. 2-3, which are simplified schematics of a full-body immobilization and transport framework 100 according to an embodiment of the present invention. FIG. 2 shows framework 100 open and ready for use, and FIG. 3 shows a patient's body enclosed in framework 100 and ready for transport.

Framework 100 shown in FIGS. 2-3 provides a comprehensive systematic approach for the emergency transportation and treatment of multiple trauma patients. The embodiment shown in the figures can be lightweight, foldable, and can easily be carried into a trauma arena or into combat in the military, e.g., on backpacks. The various straps and other means for connecting framework 100 to a patient are optionally designed for rapid and/or simple closing and/or opening, so as to minimize the time and/or number of operations required to attach a traumatized patient to framework 100 and to transport the patient to a medical facility.

Framework 100 comprises an (optionally foldable) stretcher 110 optionally having handles 41 which are of adjustable length (e.g. telescoping handles with twistable locks), so as to accommodate patients of varying body configurations. Framework 100 comprises multiple sleeves 42, and optionally comprises four sleeves 42 enabling to immobilize and protect all four limbs of a patient. Each limb may be placed into one of sleeves 42, each sleeve attached around one of the patient's limbs using attachments 80 as described above.

Framework 100 optionally comprises a pelvis fixation, strapping and anchoring element 30, groin and pelvic circumferential strapping elements 40, and adjustable members 41 which can serve as fixation points for attaching distal attachments 84 of sleeves 42 and may also serve as handles for carrying stretcher 110. In some embodiments of methods of use of framework 100, a patient's pelvis is first attached to framework 100, then sleeves 42, shoulder fixtures and head support fixtures are attached to the patient, then traction is applied to sleeves 42 as appropriate.

Framework 100 optionally provides fixtures for attaching sleeve proximal attachments 82 and sleeve distal attachments 84, enabling application of longitudinal traction to the sleeves. In some embodiments, proximal connectors 82 of sleeves 42 designed for the legs are attached at or near pelvic strapping elements 40, and proximal attachments 82 of sleeves 42 designed for the arms are attached at or near shoulder strapping elements 50. In some embodiments, these proximal attachments are positioned near the midline of the framework, so that they are under the patient when the patient is attached to the framework.

Optionally, for each sleeve 42 a longitudinal traction force element 43 is provided to facilitate applying longitudinal traction force to the sleeve. Applying such a traction force will tend to reduce fractures of the limbs, since whereas longitudinal pulling forces are evenly applied along the limb, most movement within the limb will occur at the point of fracture because the bone is discontinuous at that point and limb tissues in that area are most free to move. Traction force element 43 may be a hand crank with a ratchet arrangement controlling release of traction tension, or may be any other mechanical arrangement which facilitates applying and holding traction tension. Optionally a traction force measurement and control element 44 (also called sensor 44 herein) is provided for each sleeve, and provides a measurement or other indication of the strength of the traction forces being applied. Measurement element 44 can be a simple spring-loaded tension indicator, showing tension on a numerical scale. Alternatively, measurement element 44 can be a spring-loaded device showing different colors depending on whether the indicated tension is below, near, or above a recommended amount. Alternatively, sensor 44 can be an electronic tension-measuring device reporting by wire or wirelessly to a data terminal. Alternatively, any other tension measurement device may be used.

Sensors 44 can also be used within nets 81 to show actual tension of sleeves 41, measuring the compressive forces of sleeve 41 on a contained limb. Optionally, electronic and/or mechanical pressure sensors can be used. In some embodiments, net elements can optionally be constructed to show different colors depending on the distance of net elements on from another; that distance will indicate the level of tension in net 81 since net 81 is constructed of an elastic material.

Optionally, a pulse measurement device 120 may also be provided to report (e.g. make visible or audible or report to a wired or wirelessly connected digital device) a pulse detectable in a limb under traction. Pulse measurement device 120 can be useful because both longitudinal traction forces and induced lateral compressive forces will tend to reduce circulation in the limb. This is useful for limiting bleeding but may itself damage limb tissue if circulation is excessively inhibited. (Swelling due to fracture or other trauma can also influence blood circulation in the limb.) Pulse measurement device 120, like traction force measurement and control element 44, may optionally be provided to enable medical personnel to monitor the forces applied to the limb and/or to monitor the influence of these forces on blood circulation within the contained limb. Blood oxygenation sensors 130 may also optionally be provided and used to report on the limb oxygenation, for the same purpose.

Optionally, a feedback servomechanism can be provided so that applied traction tension may be adjusted automatically as a function of detected pressures and/or pulse strength and/or oxygenation levels and/or other relevant sensory input. For example, in some embodiments a sensor signal can be sued to control a motor or a latch on a tension element so as to increase and/or decrease tension as a function of sensor measurements, and/or according to a periodic schedule or algorithmically controlled program. In some method of use embodiments, to maximally limit bleeding a physician or automated device will increase tension until pulse and/or oxygenation sensors indicate that further tension will excessively inhibit circulation.

Framework 100 is also optionally provided with additional fixation members for attaching a patient to the framework, including for example a body, torso and head releasable connected fixation element 45 that is adjustably connected to pelvis fixation and strapping anchor element 40, a shoulder harness 50, a forehead strap 64, and a head rest and stabilization element 70, optionally provided with a chin strap, optionally operable to lock the head/neck region to the frame.

Use of Framework 100 as shown in the Figures and as described herein can provide one or more significant advantages in the treatment of severely injured patients under field conditions:
Limb fixation, traction, and compression can all be accomplished by closing a net 81 over a limb and cranking a traction force element 43, a procedure which is both simple and rapid as compared to previously known methods.
Dressing of wounds and attachment of patient to stretcher are accomplished together, rather than requiring separate acts, and can be accomplished with a minimum of movement of the patient during preparation for transportation, thereby protecting the patient from damage sometimes induced by wound dressing and attachment procedures.
Use of framework 100 also simplifies storage as compared to prior art equipment where multiple independent bandages and fixation devices are typically used. This advantage is significant in emergency vehicles where space is limited.

Framework 100, in some embodiments thereof, provides a unified system which is simple to use, requires minimal movement of the patient, is easy to store, and provides full immobilization of the body including traction for broken limbs and a high degree of control over bleeding. In some embodiments, each sleeve provides traction and bleeding control for one of the limbs, and the system as a whole firmly fixes the entire body to a transportable stretcher while constricting the tissues sufficiently to limit blood loss from the various wounds.

According to some embodiments, a method of use comprises a) approaching the framework to the trauma victim, b) attaching pelvis, shoulders and head to the stretcher, c) bandaging where necessary, d) enveloping each limb in one of the sleeves, e) providing longitudinal traction to each of the limbs as needed, and f) transporting the patient. This apparatus and method provides for faster stabilization and earlier transportation of trauma victims, as compared to the relatively more complex and time-consuming procedures of prior art. As the sleeves are pulled into axial traction they tighten around the extremities and act as pressure dressings circumferentially around the entire extremities. The axial pull also exerts moderate axial traction onto the extremities so as to reduce blood loss from extremity injuries.

Framework 100, in some embodiments thereof, provides one or more of the following functions: control of the position and stability of the entire body and of all four limbs and their alignment, control of the magnitude and direction of applied forces used to maintain or control the position of the limbs, pelvis and torso in relation to the body, reduction or partial reduction of fractures by induced traction, control of lacerations, limb, pelvis and spine fractures or open wounds to achieve the optimal clinical outcome, control of the position of the torso, pelvis and alignment of the extremities in an adjustable fashion that will fit a wide range of external body geometries, (e.g. by adjusting length of handles 41 to which distal sleeve connectors 84 are attached), and accommodation and control of the position, stability, and alignment of the body and extremities when limb or pelvis malformation exists due to fractures, open large wounds or amputations. In some embodiments continuous treatment may be administered after the patient is fixed upon the device, by opening and closing individual connectors 80, enabling emergency medical staff to open or partially open selected portions of selected sleeves 42, to access and maintain treatment of the various injuries to the body and limbs in the field and en route to the hospital.

Optionally, framework 100 may be provided with easily adjustable posts connecting between members of the device that can be used to adjust length, flexion, extension, left-right tilt, axial rotation, traction and/or compression of the different body parts and limbs in relationship to the entire body, provide a limb fixation system that has traction capabilities thus enabling fixation of long bone fractures, provide anatomically fitting circumferential limb and pelvis dressing capabilities that enable easy wound dressing (by opening and closing connectors 80) and direct wound pressure solutions (using sleeves 42, tension elements 43 and sensors 44) even in difficult-to-treat injury situations. Pelvis support that anatomically fits the patient and is applied in one or more pieces can be easily fit over the left and right groins and fastened to the body, thereby anchoring the pelvis to the device which supports the entire body and all limbs using adjustable connectors designed to fit a wide range of body types and sizes.

Optionally, framework 100, in some embodiments thereof provides head and neck support, provides an easily deployed extrication system that enables carrying the patient out of the injury scene in a safe and rapid fashion, provides anatomically fitting members to different body parts and supports that allow rapid attachment of these members to the device with connecting supports, in a variety of configurations to allow emergency personnel to stabilize the torso, pelvis, spine and extremities in the necessary position in the field, and enables attendants to adjust theses positions as medically required en route to the hospital or once the patient arrives there. Framework 100 being (optionally) transparent to x-rays, a patient arriving at a medical facility can be maintained in framework 100 while being moved (the patient together with framework 100) onto an emergency room bed, an x-ray or CT table, an operating room table, etc., thereby protecting the patient from unnecessary movement of the body parts relative to each other, and facilitating and speeding the care and treatment process.

Optionally, framework 100, in some embodiments thereof, provides a simple and convenient method for monitoring loads applied between the extremities and pelvis or body (using sensors 44) to allow emergency medical personnel and physicians to make sure that excessive forces or loads are not applied, provides facilities whereby physicians may gradually apply increasing forces or loads as needed to stop bleeding or to align fracture fragments in the extremities (using traction force element 43), and provides a device that enables the physician a simple and convenient method to accurately diagnose and assess the body, limbs, and spine for instability.

Framework 100 provides, in some embodiments thereof a simple and convenient method for accurately and safely treating multiply injured trauma survivors, enables the physician to accurately and safely reduce pelvis and limb fractures, dislocations and fracture dislocation injuries that render pelvis or limbs unstable.

Framework 100 is optionally constructed to be radiolucent, allowing accurate imaging of limbs, pelvis and spine while a patient's body is contained within and stabilized by the device. Furthermore, since framework 100 comprises multiple attachments for individual limbs and other body parts, framework 100 can stabilize and protect the injured body while allowing selective access to pelvis, limbs, spine and other body parts as required for medical treatments.

Attention is now drawn to FIG. 4, which shows a sleeve 42 or other elastic material used to control bleeding from a stump of an amputated limb or from any other pelvic wound. Framework 100 optionally comprises a sleeve 42 having a proximal attachment 82 connected to framework 100 near the region of the pelvis. In some embodiments attachment 82 connects to the frame near the midline of the pelvic carrier 30 so that attachment 82 (well padded) is under the buttocks when the patient is attached to framework 100.

In an optional embodiment shown in FIG. 4, a patient having an amputated limb is fixed to framework 100 so that a proximal connector 82 of sleeve 42 is connected under the pelvis and near the body midline. Then, as shown in FIG. 4, sleeve 42 may be extended over the amputation stump, cross the body, and be attached to framework 100 so that sleeve 42 crosses the upper pelvic brim, thereby wrapping the (bandaged) stump. Traction may optionally be applied to the sleeve 42 as described above, thereby applying pressure to the stump bandages to control bleeding.

Bleeding from a cavity wound, such as a traumatic cavity in the pelvis, can be similarly treated. After pads or compresses are provided to fill the cavity, a sleeve 42 whose proximal attachments 82 are attached under the pelvis can be extended across the cavity wound, across the body, and to the framework, and traction can be applied as described above, thereby pressing sleeve 42 onto the bandages filling the cavity wound, thereby controlling bleeding.

It is noted that although the preceding description of FIG. 4 mentions use of framework 100 and the use of a sleeve 42, the embodiment shown in FIG. 4 can be accomplished using any elastic or even an inelastic material under tension. For controlling bleeding of a stump of an amputated leg or bleeding from a cavity wound of the pelvis, the material used need not necessarily be formed as a sleeve.

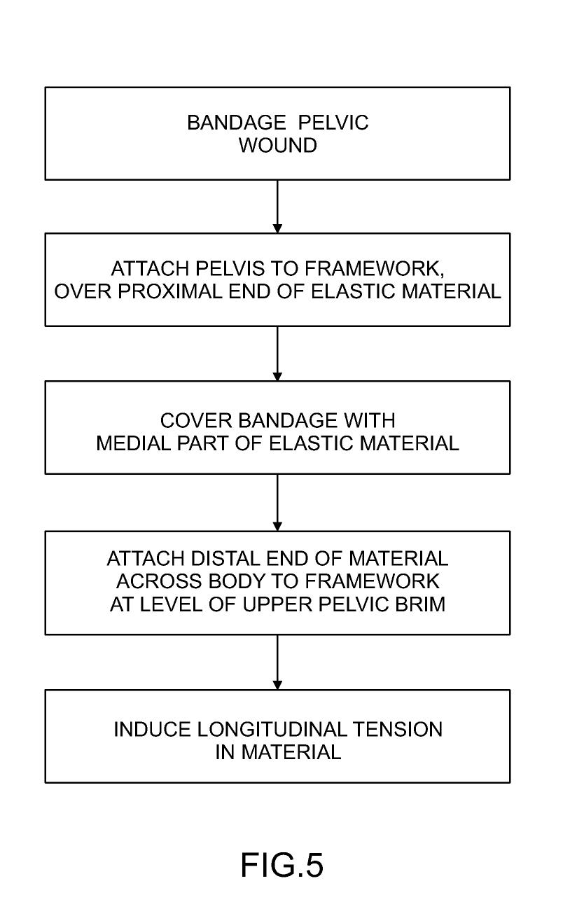

Attention is drawn to FIG. 5, which is a flowchart of the method sown in FIG. 4, according to an embodiment of the present invention. The flowchart includes:
Bandaging the pelvic wound, packing the wound if appropriate;
Attaching patient's body to framework which has an elastic material attached to that framework at the level of the pelvis and near the midline of the body;
Using a medial part of the elastic material to cover the pelvic bandage
Attaching a distal end of material across the body to the framework, so that the material falls across the upper pelvic brim; and
Inducing longitudinal tension in the material sufficient to control bleeding.

It is expected that during the life of a patent maturing from this application many relevant immobilization devices will be developed and the scope of the terms “immobilization device”, “framework”, “stretcher”, and “sleeve” are intended to include all such new technologies a priori.

The terms “comprises”, “comprising”, “includes”, “including”, “having” and their conjugates mean “including but not limited to”.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device for stabilizing, stretching, and compressing a wounded limb of a patient, comprising
a) an elastic net which comprises
i) a plurality of lateral fasteners which, when fastened, shape said net into a sleeve which may be fastened around said limb;
ii) at least one proximal fastener on a proximal end of said sleeve; and
iii) at least one distal fastener on a distal end of said sleeve, said net being so constructed that when a limb is encased in said net by fastening said lateral fasteners and longitudinal traction of between 5 kg and 20 kg is applied to said net in a direction along a length of said limb by pulling said proximal and said distal fasteners away from each other, sufficient lateral pressure is applied toward said limb by said net to reduce bleeding from said limb.

2. The device of claim 1, further comprising
b) a framework sufficiently strong to support at least a limb of a body, and to which said proximal and distal fasteners may be attached.

3. The device of claim 1, further comprising a tension device for providing a traction tension by pulling said proximal and said distal fasteners away from each other, said tension being adjustable to a range at least partly overlapping with the range of between 5 kg and 20 kg of tension.

4. The device of claim 2, wherein said framework is a stretcher.

5. The device of claim 4, further comprising four of said nets, two sized to form sleeves each sized accommodate an arm, and two sized form sleeves each sized to accommodate a leg.

6. The device of claim 4, wherein said stretcher comprises at least one extended handle whose length is adjustable.

7. The device of claim 2, where said framework comprises a Thomas splint.

8. A full-body transportation apparatus for transporting a wounded patient, which comprises
a) a stretcher which comprises a pelvic attachment for attaching a pelvis of a patient to said stretcher;
b) a plurality of nets each of which comprises
i) an elastic body;
ii) lateral fasteners which shape said material nets into a sleeve which may be fastened around a limb of a patient;
iii) proximal and distal fasteners attachable to portions or fixtures of said stretcher; and
iv) a tension controller operable to introduce a controllable degree of longitudinal traction to said each sleeve,
and wherein at least some sleeves formed of said nets are so constructed that introducing longitudinal traction forces on at least one sleeve formed from at least one of said nets produces lateral constricting pressures on a limb contained within said sleeve.

9. The stretcher of claim 8, further comprising a pressure sensor for measuring longitudinal traction applied to at least one of said nets formed as a sleeve and enclosing a limb of a patient.

10. The stretcher of claim 8 or 9, further comprising a pressure sensor for measuring lateral constricting pressure applied to an enclosed limb by at least one of said nets formed as a sleeve.

11. The stretcher of claim 8, further comprising a pulse sensor operable to detect and report strength of a pulse on a distal portion of a limb under traction by one of said nets formed as a sleeve and enclosing said limb.

12. The stretcher of claim 8, further comprising a blood oxygenation detector operable to detect blood flow status on a distal portion of a limb under traction by one of said nets formed as a sleeve and enclosing said limb.

13. The stretcher of claim 8, further comprising telescoping handles each of which may be fixed to be of a selected length, said handles comprising a feature to which a distal attachment of one of said nets may be attached.

14. The stretcher of claim 8, wherein said lateral constricting pressures on a limb contained within a sleeve is sufficient to reduce bleeding from said limb.

15. A method for reducing a fracture of a limb, comprising
a) enclosing said fractured limb in an net; and
b) creating a traction tension by pulling a distal end of said net away from a proximal end of said net, thereby elongating said net, causing said net to apply pressure to said enclosed limb and to transfer at least some of said traction to said limb, thereby reducing said fracture.

16. The method of claim 15, wherein said pressure applied by said net to said enclosed limb is sufficient to reduce bleeding from said limb.

17. The method of claim 15, wherein said traction tension is regulated to a tension of between 5 kg and 20 kg.

18. The method of claim 15, further comprising using a pressure sensor to report traction tension induced in said net.

19. The method of claim 18, further comprising adjusting traction tension in said net so as to maximize tension without reducing circulation in said limb to a dangerous degree.

20. The method of claim 19, further comprising utilizing one of a pulse detector and a blood oxygenation detector to detect dangerous reduction in blood circulation of said limb.

21. A method for controlling bleeding from a pelvic wound of a patient during transportation of said patient, comprising:
a) applying bandaging materials in or on said pelvic wound;
b) attaching said patient to a framework having a proximal end of an elastic material attached to said framework near a midline of said framework and near a feature designed to receive and bind a pelvis of a patient to said framework;
c) positioning a medial portion of said material so that it at least partially covers said bandaging material;
d) attaching a distal end of said elastic material to said framework in a manner which causes said material to substantially cover said bandages, cross over said patient's body, pass near an upper pelvic brim of said patient, and be attached to said framework; and
e) inducing a longitudinal tension in said material, thereby pressing said bandaging material toward said pelvic wound to inhibit bleeding therefrom.

22. A method for reducing bleeding from a wounded limb, comprising
a) enclosing said wounded limb in an net; and
b) creating a traction tension by pulling a distal end of said net away from a proximal end of said net, thereby elongating said net, causing said net to apply pressure to said enclosed limb, said pressure being sufficient to reduce bleeding from said limb.
